(19) United States
(12) Patent Application Publication
(10) Pub. No.: US 2004/0253156 A1
(43) Pub. Date: Dec. 16, 2004

(54) Machine system for sterilising and filling containers

(76) Inventors: Werner Stahlecker, Goppingen (DE); Peter Awakowicz, Munchen (DE); Robert Frost, Landshut (DE)

Correspondence Address: CROWELL & MORING LLP, INTELLECTUAL PROPERTY GROUP, P.O. BOX 14300, WASHINGTON, DC, 20044-4300, US

(21) Appl. No.: 10/486,699

(22) PCT Filed: Jul. 18, 2002
(86) PCT No.: PCT/EP02/07991

(30) Foreign Application Priority Data
Aug. 15, 2001 (DE) 101 40 807.2

(51) Int. Cl. A61L 2/20

(52) U.S. Cl. 422/302

(57) ABSTRACT

A cyclical functioning machine installation for sterilizing and filling containers comprises a sterilization chamber, to which is placed a feeding device arranged in an non-sterile hold for the joint feeding of a given number of containers to be sterilized, and to which sterilization chamber is placed a delivery device for the joint removal of sterilized containers. According to the present invention, the delivery device is arranged in a sterile hold, which is separated from the feed device by the sterilization chamber which acts as a lock. A filling device may also be arranged in the sterilization chamber.

This application is a National Phase of international patent application no. PCT/EP 02/07991, filed Jul. 18, 2002, the entire disclosure of which is incorporated herein by reference. Priority is claimed based on German patent application no. 101 40 807.2, filed Aug. 15, 2001.

### BACKGROUND AND SUMMARY OF THE INVENTION

The present invention relates to a cyclical functioning machine installation for sterilizing and filling containers, comprising a sterilization chamber, also comprising a filling device, also comprising a feed device arranged in an non-sterile hold for the joint feed of a given number of containers to be sterilized in the sterilization chamber as well as with a delivery device for the joint removal of the sterilized containers from the sterilization chamber and for the continued transport of the containers.

Machine installations of this type are known in the industry. The feed device comprises a transport belt which transports in a first cycle the containers to be sterilized, for example bottles, from a container feed into a non-sterile hold adjacent to the sterilization chamber. In a subsequent cycle, the containers standing in readiness on the transport belt are simultaneously transferred into holding devices of a lowered loading floor of the sterilization chamber. The loading floor subsequently travels upwards into the sterilization chamber, and the chosen sterilization process begins. When the sterilization process is completed, the loading floor is lowered again. In the subsequent cycle the sterilized containers are removed from the loading floor and transferred to a second transport belt which is a part of the delivery device. The second transport belt brings the just sterilized containers into a filling chamber. In a further cycle, these containers are then pushed simultaneously in a parallel displacement under filling valves and filled. Subsequently, the filled containers are removed from the filling stations and transferred to a transport belt disposed opposite thereto, which transport belt then guides the containers to the sealing stations.

In the known machine installations, there is a constant connection between the feed device and the delivery device, and the non-sterilized containers of the feed device are therefore located in the same space as the sterilized container of the delivery device. In an attempt to avoid recontamination, appropriate current ratios have been created, but as there is no hermetic separation of the non-sterile and the sterile areas, there is always the risk of contamination in the known machine installation.

It is an object of the present invention to eliminate the risk of contamination in the known machine installations.

This object has been achieved in accordance with the present invention in that the delivery device is arranged in a sterile hold, which is separated from the feed device by means of a sterilization chamber which acts as a material lock.

The sterilization chamber can take the form of a lock between the feed device and the sterile hold in that it is bordered on its sides by partition walls which can be opened and closed independently of each other, and which advantageously take the form of lifting or lowering partitions. Thus the sterilization chamber is hermetically sealed off from the sterile hold when the containers are fed, and from the feed device when the sterilized containers are being removed. It can be additionally provided that the filling device is arranged directly in the sterilization chamber.

These and further objects, features and advantages of the present invention will become more readily apparent from the following detailed description thereof when taken in conjunction with the accompanying drawings wherein:

### BRIEF DESCRIPTION OF THE DRAWINGS

FIG. 1 is a top view of a machine installation and of the type of feed and delivery of a group of containers in transverse direction to the machine installation,

FIG. 2 is an intersection along the intersectional surface II-II of FIG. 1,

FIGS. 3A to 3F show in schematic form the individual paths travelled by the containers during sterilization,

FIG. 4 is a schematic and perspective depiction of the machine installation, its feed and delivery devices being in longitudinal direction of the machine installation.

### DETAILED DESCRIPTION OF THE DRAWINGS

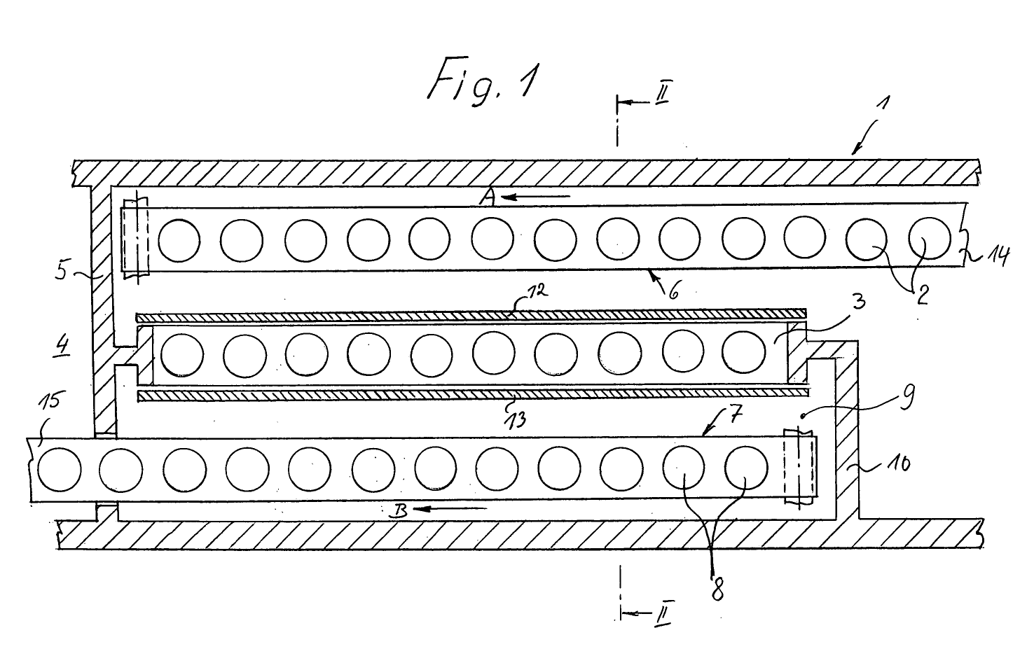

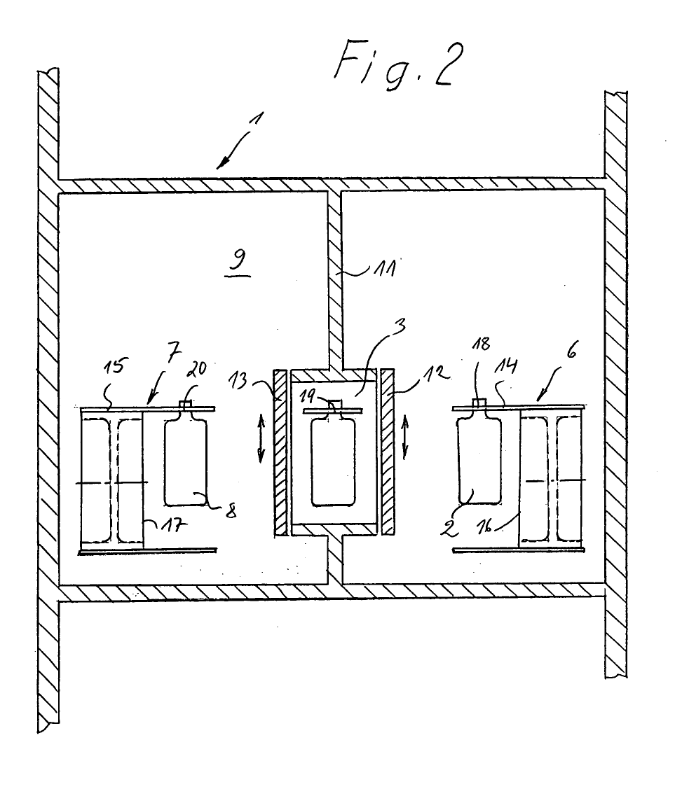

The machine installation 1 shown schematically in FIGS. 1 and 2 is a cyclic functioning linear conveyor which serves to sterilize, fill and seal containers 2. The machine installation 1 comprises among others a sterilization chamber 3 which can be evacuated, downstream of which sterilization chamber 3 in longitudinal direction of the machine installation 1 a filling chamber 4 is arranged together with other chambers (not shown). The filling chamber 4 is separated from the sterilization chamber 3 by a partition wall 5.

The machine installation 1 comprises a feed device 6 arranged in a non-sterile hold for the containers 2 to be sterilized. The feed device 6 is located in transverse direction to the machine installation 1 adjacent to the sterilization chamber 3, said feed device 6 comprising a transport belt 14 which travels in feed direction A. The containers 2 to be sterilized are placed laterally adjacent to the sterilization chamber 3 in readiness and can be jointly transferred to the sterilization chamber 3 from the side. The number of containers 2 to be sterilized collectively is predetermined and is shown in FIG. 1 as a group of ten containers 2.

The delivery device 7, which comprises a transport belt 15 which travels in the direction of motion B is arranged on the side of the sterilization chamber 3 facing away from the feed device 6 and serves to remove the sterilized containers 8 collectively from the sterilization chamber 3. These sterilized containers 8 are then fed in delivery direction B to the filling chamber 4.

The section described above is known in principle from assembled machine installations and has the disadvantage that the feed device 6 and the delivery device 7 are arranged in principally the same space, which is detrimental to a full aseptic environment.

It is a feature of the present invention that the delivery device 7 is completely hermetically sealed off from the feed device 6 and arranged in a sterile hold 9. The sterile hold 9 is separated from the outer environment by means of partition walls 10 and 11, and the sterilization chamber 3 functions as a kind of lock in relation to the feed device 6. This lock works in a particular way, as the interior of the sterilization chamber 3 is connected alternately with the feed device 6 and then with the delivery device 7.

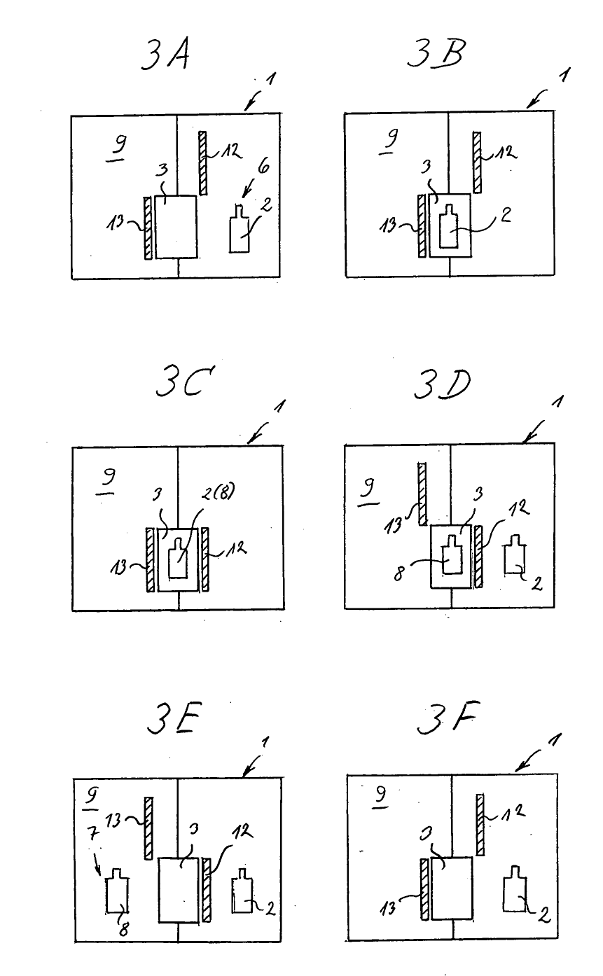

The sterilization chamber 3 can be hermetically sealed laterally by means of partitions 12 and 13, which can be moved independently of one another. These partitions 12 and 13 can be moved in the directions denoted by the double arrows in FIG. 2 and are capable of sealing off the sterilization chamber 3 from the outside during sterilization due to their movements. The partitions 12 and 13 can be raised and lowered, as described below with the aid of FIG. 3.

It should be mentioned here that the transport belts 14 and 15 are guided over suitable guiding wheels 16 and 17, and that the transport belts 14 and 15 comprise suitable holding devices 18 and 20 for the containers 2 or 8. In a similar way, holding devices 19 are provided in the sterilization chamber 3 for the containers 2 to be sterilized.

As mentioned above, the type of sterilization in the sterilization chamber 3 in connection with the present invention can be any preferred method. It is particularly practical, however, to carry out the sterilization using hydrogen peroxide, which is fed in a watery solution to an evaporator, vaporized and subsequently guided into the evacuable sterilization chamber 3, where a steam formed of water and hydrogen peroxide is condensed on the surfaces of the containers 2 to be sterilized and on the sterilization chamber 3, and which thus effects a rapid sterilization. During condensation, evaporation enthalpy is freed, which delivers the necessary energy to dissociate a hydrogen peroxide molecule so that an oxygen atom is released. This highly reactive chemical atomic oxygen is, in the opinion of the applicant, responsible for the sterilizing effect, whereby it is sufficient when the surface to be sterilized is covered with a microscopically thin homogenous liquid film during condensation. The condensate can be suctioned off by means of pumps (not shown). This sterilization method also has the great advantage that the sterilization chamber 3 is freed anew of bacteria during each sterilization process.

The interplay of the sterilization chamber 3 with the feed device 6 and the delivery device 7 is described below with the aid of FIGS. 3A to 3F, whereby FIG. 3F shows the end of one cycle and is identical to FIG. 3 which shows the beginning of the cycle.

The FIGS. 3A to 3F show in particular that the sterilization chamber 3 is hermetically sealed off from the sterile hold 9 when the containers 2 are being transferred in and hermetically sealed off from the feed device 6 when the sterilized containers 8 are being removed.

The FIG. 3A shows the state of the machine installation 1 in which a group of containers 2 to be sterilized are held in readiness laterally adjacent to the sterilization chamber 3 by means of the feed device 6. The partition 12 between the sterilization chamber 3 and the feed device 6 is, as can be seen, raised, so that a direct contact between the feed device 6 and the sterilization chamber 3 exists. The partition 13 seals the sterilization chamber 3 off laterally in relation to the sterile hold 9, in which the delivery device 7 is located.

FIG. 3B shows how the group of containers 2 to be sterilized are fed transversely to the sterilization chamber 3 into same, whereby the partition 12 is still raised. In FIG. 3B, where the feed device 6 is no longer shown, there are no containers 2 present.

According to FIG. 3C, both partitions 12 and 13 seal off the sterilization chamber 3 laterally, so that the sterilization chamber 3 is closed in a sealed manner in relation to the feed device 6 as well as to the sterile hold 9 in which the delivery device 7 is located. The group of containers 2 is now rendered free of bacteria in the closed off sterilization chamber 3, so that subsequently sterilized containers 8 are present.

In FIG. 3D, the partition 13, which closes off the sterilization chamber 3 to the sterile hold 9, is raised in the way shown, while the other partition 12 continues to seal off the sterilization chamber 3 from the feed device 6 and from the new containers 2 which stand therein adjacent to the sterilization chamber 3 in readiness. The sterilized containers 8 can thus be laterally removed by the delivery device 7 located in the sterile hold 9.

The state in FIG. 3E shows that the group of sterilized containers 8 have been transferred laterally out of the sterilization chamber 3 onto the delivery device 7. When the partition 13 arranged to the sterile hold 9 seals off the sterilization chamber 3 again by being lowered, and the other partition 12 arranged to the delivery device 6 is raised, the state at the beginning of the process as shown in FIG. 3A arises, as shown again here in FIG. 3F.

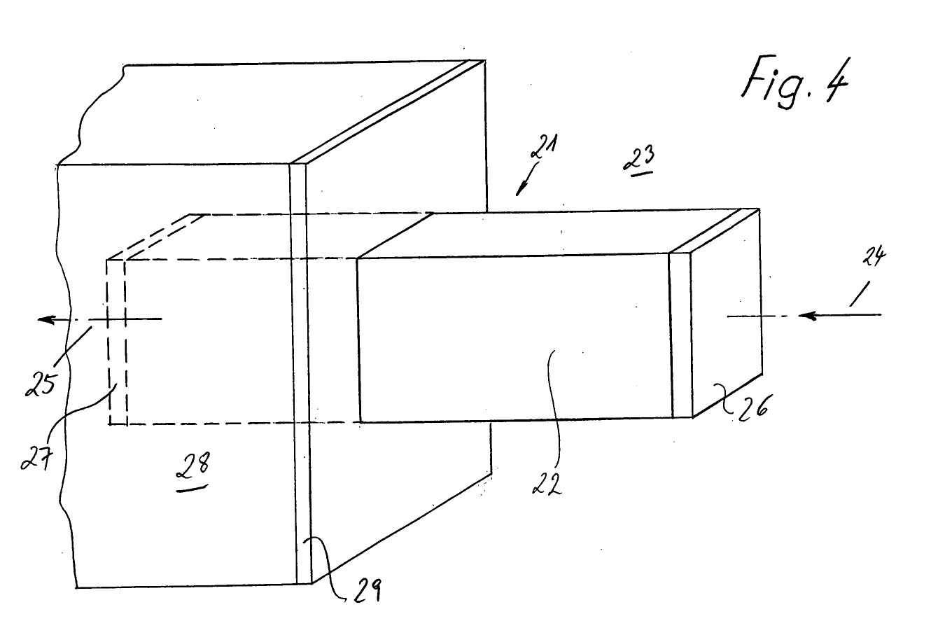

In the case of the machine installation 21 according to FIG. 4, the feed device 24 and the delivery device 25 are separated in longitudinal direction of the machine installation 21 by the sterilization chamber 22, which may also hold the filling device. The sterile hold 28 is also separated from the feed device 24 arranged to the non-sterile space 23 by the sterilization chamber 22 which acts as a lock, and by a partition wall 29. The sterilization chamber 22 comprises an entry door 26 preferably in the form of a flap, as well as an exit door 27.

The foregoing disclosure has been set forth merely to illustrate the invention and is not intended to be limiting. Since modifications of the disclosed embodiments incorporating the spirit and substance of the invention may occur to persons skilled in the art, the invention should be construed to include everything within the scope of the appended claims and equivalents thereof.

1. A cyclical functioning machine installation for sterilizing and filling containers, comprising:
a sterilization chamber;
a feed device arranged in an non-sterile hold for the joint feed of a plurality of containers to be sterilized in the sterilization chamber;
a delivery device for the joint removal of the sterilized containers from the sterilization chamber and for the continued transport of the containers,
wherein the delivery device is arranged in a sterile hold which is separated from the feed device by the sterilization chamber which acts as a lock.

2. A machine installation according to claim 1, further comprising:
a filling device, wherein the filling device is arranged in the sterilization chamber.

3. A machine installation according to claim 1, wherein the feed device and the delivery device are separated in a longitudinal direction of the machine installation (by the sterilization chamber and are divided by a partition wall from one another.

4. A machine installation according to claim 1, wherein the feed device and the delivery device are separated in a transverse direction of the machine installation by the sterilization chamber and divided by a partition wall.

5. A machine installation according to claim 4, wherein the sterilization chamber is bordered on both sides by partitions which open and close independently of one another.

6. A machine installation according to claim 5, wherein the partitions are in the form of partitions which can be raised and lowered.

7. A machine installation according to claim 4, wherein the sterilization chamber is hermetically sealed off in relation to the sterile hold while the containers are being transported in, and in relation to the feed device when the sterilized containers are being removed.

8. A machine installation according to claim 5, wherein the sterilization chamber is hermetically sealed off in relation to the sterile hold while the containers are being transported in, and in relation to the feed device when the sterilized containers are being removed.

9. A machine installation according to claim 6, wherein the sterilization chamber is hermetically sealed off in relation to the sterile hold while the containers are being transported in, and in relation to the feed device when the sterilized containers are being removed.

10. A machine installation according to claim 2, wherein the feed device and the delivery device are separated in a longitudinal direction of the machine installation by the sterilization chamber and are divided by a partition wall from one another.

11. A machine installation according to claim 2, wherein the feed device and the delivery device are separated in a transverse direction of the machine installation by the sterilization chamber and divided by a partition wall.

12. A machine installation according to claim 10, wherein the sterilization chamber is bordered on both sides by partitions which open and close independently of one another.

13. A machine installation according to claim 11, wherein the partitions are in the form of partitions which can be raised and lowered.

14. A machine installation according to claim 11, wherein the sterilization chamber is hermetically sealed off in relation to the sterile hold while the containers are being transported in, and in relation to the feed device when the sterilized containers are being removed.

15. A machine installation according to claim 12, wherein the sterilization chamber is hermetically sealed off in relation to the sterile hold while the containers are being transported in, and in relation to the feed device when the sterilized containers are being removed.

16. A machine installation according to claim 13, wherein the sterilization chamber is hermetically sealed off in relation to the sterile hold while the containers are being transported in, and in relation to the feed device when the sterilized containers are being removed.
